(19) United States
(12) Patent Application Publication
(10) Pub. No.: US 2006/0121016 A1
(43) Pub. Date: Jun. 8, 2006

(54) Methods and compositions for treatment of free radical injury

(76) Inventor: Raphael C. Lee, Chicago, IL (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/252,999

(22) Filed: Oct. 18, 2005

Related U.S. Application Data
(60) Provisional application No. 60/619,432, filed on Oct. 18, 2004.

(51) Int. Cl. A61K 31/7076; A61K 31/7048; A61K 38/05; A61K 38/43; A61K 38/44; A61K 31/385; A61K 31/353; A61K 31/198

(52) U.S. Cl. 424/94.1; 514/18; 514/47; 514/27; 514/440; 514/456; 514/562; 424/94.4; 424/702

(57) ABSTRACT

Therapeutic methods and compositions useful for the prevention and/or treatment of cellular membrane damage leading to or resulting from peroxidation of the cellular membrane and a breakdown of the barrier function of the cellular membrane. A therapeutic composition includes a combination of a membrane sealing sealing surfactant and a cofactor treatment consisting of an antioxidant and a cellular energy store. To affect this goal, the permeability of damaged cellular membranes is reestablished by the membrane sealing surfactant, effectively “sealing” the injured membranes. To facilitate rapid tissue recovery, cellular energy levels can be reestablished through addition of a cellular energy source such as, for example, MgCl₂-ATP which, serves a further dual benefit of improving the cellular ion balance. Addition of an antioxidant eliminates the generation of Reactive Oxygen intermediates and enhances the metabolism of free radicals.

### PRIORITY CLAIM

The present application claims priority to U.S. Provisional Application No. 60/619,432, filed Oct. 18, 2004, and entitled, “METHODS AND COMPOSITIONS FOR TREATMENT OF ISCHEMIS/REPERFUSION INJURY,” which is herein incorporated by reference to the extent not inconsistent with the present disclosure.

### FIELD OF THE INVENTION

The present invention relates generally to critical care medicine for the prevention or amelioration of tissue damage associated with cellular membrane injuries. More particularly, it relates to compositions and use of therapeutic compositions of membrane sealing surfactants, cellular energy sources and antioxidants for increasing the viability of mammalian cells exposed to events leading to cellular membrane peroxidation and consequently, cell death.

### BACKGROUND OF THE INVENTION

In mammalian cells that are generally considered healthy cells, the cellular membrane functions as a diffusion barrier against ion transport into and out of the cell. When healthy cells are exposed to systemic or outside events causing the cellular membrane to become permeable, the barrier function of the membrane is compromised allowing for mutual diffusion of ions across the membrane such that the metabolic energy of the cell can be quickly exhausted. As the cellular energy is depleted, the cell proceeds to biochemical arrest and eventually to cellular necrosis as illustrated generally in FIG. 1. Cellular membrane permeabilization is a common cause for tissue necrosis in a variety of tissue injuries including: (1) ischemia-reperfusion injuries, such as, for example, myocardial infarction, cerebrovascular stroke, cerebral palsy from difficult childbirth, and testicular torsion; (2) electrical injuries; (3) burns and frostbite; and (4) radiation exposure. (Hannig and Lee, 2000.)

Ischemia/reperfusion injury is relevant to many fields of human and veterinary medicine. Ischemia/reperfusion (I/R) injury occurs following every successful balloon angioplasty, tPA induced thrombolysis and organ transplant. For example, 20-30% of renal transplants fail due to acute renal failure of the graft, and more than one-half of potentially donated kidneys are not transplanted due to injury associated with hypotension. In plastic surgery, I/R injury threatens the integrity of every flap. I/R injury may follow decompression fasciotomy for a compartment syndrome, occur after the reattachment of a severed extremity or occur following the release of testicular torsion. Successful resuscitation of critically ill patients can result in a multiorgan failure syndrome in which reperfusion injury plays a critical role. Finally, after colic surgery, the return of blood and oxygen to a previously strangulated segment of intestine causes I/R injury in the affected area which contributes to many of the post-operative complications that can cause death in horses. Clearly, there is a vital need to reduce I/R injury through the development of more effective therapies.

When tissue is subjected to ischemia, a sequence of chemical events is initiated that may ultimately lead to cellular dysfunction and necrosis. If ischemia is ended by the restoration of blood flow, i.e., by reperfusion, a second series of injurious events ensue producing additional injury. Thus, whenever there is a transient decrease or interruption of blood flow, the net injury is the sum of two components—the direct injury occurring during the ischemic interval and the indirect or reperfusion injury, which follows. Animal models have shown that, at least within the first minutes to hours after the onset of ischemia, the ultimate fate of tissue after reperfusion is dependent upon the duration and the depth of hypoperfusion (Jones et al., 1981, 1994). For example, the intestinal injury induced by 3 hours of ischemia (flow reduced to 20% of normal) and one hour of reperfusion is several times greater than that observed after 4 hours of ischemia alone (Parks and Granger, 1986). This same pattern of relative contribution of injury from direct and indirect mechanisms has been shown to occur in all organs.

Most studies of cerebral blood flow in animal models have consistently shown that reperfusion within 3 hours of arterial occlusion will limit to some extent the size of the resulting infarct and improve other measures of outcome as well (Jones et al., 1981; Kaplan et al., 1991). These studies also show, however, that reperfusion after the 3 hour time point will have little or no benefit or may make things worse (Yang and Betz, 1994). In fact, understanding the pathophysiology of such “reperfusion injury” now assumes greater importance since some patients treated with t-PA even within the 3 hour time window will develop cerebral edema and/or hemorrhage (Hacke et al., 1995), and others may harbor less obvious consequences of reperfusion at the cellular level which negate the benefits of reestablishing adequate blood flow.

In vivo and in vitro model systems of cerebral ischemia have provided some understanding of the ischemic cascade. The cascade, which starts with the reduction of cerebral blood flow, is rapidly followed by inhibition of protein synthesis, depletion of intracellular energy stores, and membrane depolarization. Membrane depolarization causes opening of voltage-operated calcium channels allowing disruption of tightly regulated neuronal calcium homeostasis. Glutamate is released from presynaptic stores and, in the presence of glycine, activates the N-methyl-D-aspartase (NMDA) receptor. The immediate consequence is increased sodium permeability and cellular swelling, but the more damaging event is further elevation of intracellular calcium. Further perturbations in ion flux occur as a result of glutamate's effect on the adenosine monophosphate and metabotrophic receptors.

Increased intracellular calcium activates a large number of damaging enzymatic pathways, including protein kinases, proteases, and lipases. The consequences of nitric oxide, free-radical production, and these enzyme perturbations are widespread, including disruption of neuronal and endothelial membranes, cytoskeletal integrity, and damage to mitochondrial function. It is generally accepted that massive calcium influx or calcium overload during the first minutes of reperfusion leads to the destruction of the sarcolemma and subsequent cell death. Thus, during 60-90 minutes of ischemia, the sarcolemma is altered in such a way that the barrier function for calcium is lost. Several scenarios have been proposed to explain the changes of the sarcolemma during ischemia, including changes in phospholipids asymmetry by ATP depletion, oxygen free radical formation, formation of arachidonic acid by phopholipase A2 and fatty acid accumulation by the lack of β-oxidation and a decrease of pH.

The reactions initiated at reperfusion involve the formation of cytotoxic oxidants derived from molecular oxygen. During an ischemic episode variable amounts of hypoxanthine are produced. Reperfusion provides oxygen to the post-ischemic tissues. The reaction of molecular oxygen with xanthine oxidase in the presence of hypoxanthine yields highly reactive free radicals which appear to play a major role in I/R injury of the small intestine (Parks et al., 1982).

It appears that the mechanism of intestinal I/R injury is multifactorial, involving not only reactive oxygen metabolites, but also luminal proteolytic enzymes, neutrophils, nitric oxide, endothelia, prostaglandins and other unidentified agents. Recently, reduced nitric oxide production (Mueller et al., 1994) and neurophil activitation (Gonzalez et al., 1994) have been shown to be associated with intestinal I/R injury and endothelial damage. Neutrophils contain an NADPH oxidase that reduces molecular oxygen to the superoxide anion and are the primary mediators of reperfusion induced increases in microvascular permeability.

I/R injury has also been observed to correlate with increased gene expression in ischemic regions resulting in tissue inflammation and in white blood cell interaction with vascular endothelium to produce blood brain barrier damage and plugging of the microcirculation which results in occlusion.

Numerous preclinical studies of focal ischemia in animal models have shown efficacy by targeting each of the steps along the ischemic cascade to prevent the generation of free radicals and/or enhance the capacity of a tissue to metabolize free radicals. Because drugs can interrupt the ischemic cascade in tissue that is not yet dead, they have been shown to be most effective in animal models of focal cerebral ischemia where there is an extensive ischemic penumbra or area of relatively mild ischemic injury. As their effect is primarily on penumbral regions, relatively modest benefit can be expected from using any one of these drugs alone. In acute animal models, it was found that neuroprotective therapies started after the onset of ischemia but prior to reperfusion can augment the beneficial effect of reperfusion and extend the time window for starting reperfusion therapy. None of the drugs when used alone substantially reduces infarct volume unless started within the first few hours after onset of ischemia. Further, the effectiveness of these drugs may vary from one tissue to another.

A number of antioxidants and free radical scavengers have been investigated in the prevention of I/R injury but results have been inconsistent. Intestinal injury has been prevented by various antioxidants (Parks et al., 1982; Granger et al., 1986; Nalini et al., 1993). Lazaroids have been used to protect against I/R injury of the central nervous system (Hall et al., 1988), heart (Levitt et al., 1994), lung (Aeba et al., 1992), liver (Cosenza et al., 1994) and kidney (Shackleton et al., 1994). But results of the use of lazaroids in cases of intestinal ischemia have been conflicting. Some investigators have found amelioration of mucosal injury with lazaroids (Stone et al., 1992; Katz et al., 1995), whereas others have found no protection (Park et al., 1994; Van Ye et al., 1993). These inconsistencies may be caused by differences in ischemic time, experimental model, lazaroid compound and the timing and method of drug administration.

In some animal models of reperfusion injury, the free radical scavenger superoxide dismutase (SOD) has shown promise (Flaherty, 1991), but it was ineffective in others (Vanhaecke, 1991; Euler, 1995). Clinical trial results have also been variable. Pollac et al. (1993) administered SOD or placebo as a bolus before reperfusion of transplanted kidneys and as an infusion for an additional hour, but there was no difference in post-operative renal function. In another study, a similar dose of SOD was administered as a single rapid infusion before reperfusion or renal transplants (Land et al., 1994). The incidence of acute rejection was greatly reduced and long term graft survival was enhanced. This was attributed to a reduction in free radical damage and consequently less stimulation of the immune system by the graft. Conversely, a trial of SOD in 120 patients undergoing angioplasty for acute myocardial infarctions (Flaherty et al., 1994) found no beneficial effect of the enzyme on cardiac function.

Aspirin, which inhibits platelet aggregation, has been used with great success in the reduction of ischemic injury in several organ systems. Aspirin-treated animals had a marked reduction of the gross hemorrhagic discoloration and vascular congestion seen in the untreated ischemic animals. Also, histological evaluation revealed the preservation of seminiferous tubular integrity in the aspirin-treated animals compared to the untreated animals. No marked difference was noted in the gross or microscopic finding whether aspirin was administered prior to or during the ischemic event (Palmer et al., 1997). In humans, the effect of aspirin on platelets is almost immediate depending on the rate of absorption.

Neutrophil accumulation initiated by reperfusion is significantly reduced by pretreatment with xanthine oxidase inhibitors oxygen radical scavengers or iron chelators, suggesting that reactive oxygen metabolites play a role in the recruitment of neutrophils into post-ischemic tissue and that control of neutrophil activity appears to be an important juncture for reducing reperfusion injury. But the outcomes in clinical trials have not been universally successful. Parmley et al. (1992) found more infarct extensions with allopurinol, a xanthine oxidase inhibitor, than with a placebo, contrary to expectations. Yet in coronary artery bypass grafting, lipid peroxidation was reduced by allopurinol (Coghan et al., 1994), and in other studies the incidence of complication following surgery was reduced 70% by administering allopurinol both before and after the operation (Rashid and Goran, 1991).

It is unclear whether ischemic tissue is fatally injured during reperfusion, or whether reperfusion simply unmasks injury that has already occurred. But results do indicate that reperfusion injury may be fatal to previously viable cells during ischemia/reperfusion. Neuroprotective therapy targeting neurotransmitter release and intracellular calcium-mediated events must be started very early after focal ischemia (the exact time window is unknown but none of these strategies has been effective in reducing infarct volume after middle cerebral artery occlusion in animals when started beyond 1-2 hours after the onset of ischemia), so pre-hospital treatment or prophylactic therapy of high-risk patients (i.e., those scheduled to receive coronary artery bypass or carotid endarterectomy) needs to be improved.

When biomaterials are exposed to radiation, damage to the cellular membrane can result from directly ionizing radiation (exposure to alpha, beta and neutron particles) or from indirectly ionizing radiation (exposure to ultra-violet and x-rays, gamma irradiation) as illustrated in FIG. 2. Regardless of the radiation source, ionizing radiation can lead to cellular membrane damage either through the formation of toxic fee radicals which separately attacks the cellular membrane as illustrated in FIG. 3, or through to a minor degree, direct ionization of the molecular bonds.

In addition to cellular membrane damage induced by I/R injuries and radiation exposure, the cellular membrane can suffer mechanical disruption as experienced with the disease muscular dystrophy. This mechanical disruption of the cellular membrane similarly destroys the barrier function of the cellular membrane resulting in the formation of free radicals, which further contribute to the injury.

In addition to membrane damaged induced by I/R injuries, radiation exposure and mechanical disruption, similar cellular membrane damage has been found to result from a variety of other mechanisms including electrical injury, thermal injury such as burns or frostbite, physiological conditions such as cerebral palsy, physical injuries such as spinal cord and head injuries, organ transplantation, necrotizing endocolitis, bacterial translocation and conditions characterized by exposure to chemical oxidants.

Regardless of the cellular injury mechanism, it is clear that the result is a complex series of interactions between biochemical and metabolic processes which, if unchecked, result in cellular necrosis. Although a number of singular and combinatorial therapies have been used to treat cellular membrane peroxidation, no therapy has proven to consistently alleviate the damage to the cellular membrane.

### SUMMARY OF THE INVENTION

The present disclosure relates to therapeutic methods and compositions useful for the prevention and/or treatment of cellular membrane damage comprising reduction of cellular membrane permeability, reduction of cellular peroxidation, and replenishment of cellular energy stores. The methods and compositions disclosed herein can be utilized to increase mammalian cell viability and survivability for a variety of injuries resulting in a breakdown of the barrier function of the cellular membrane. The methods and compositions disclosed herein are specifically contemplated for use in treating and preventing damage associated with cellular membrane injury as a result of systemic and outside events such as, for example, mammalian cells exposed to events such as colic, acute myocardial infarction, ischemia/reperfusion injury, cerebral palsy, muscular dystrophy, stroke, spinal cord injury, head injury, organ transplantation, necrotizing endocolitis, bacterial translocation, conditions characterized by exposure to ionizing radiation and conditions characterized by exposure to chemical oxidants which produce excess reactive oxygen species, all of which can lead to cellular membrane peroxidation and consequently, cell death.

An illustrative system for the prevention or treatment of ischemia/reperfusion injury can comprise administering to tissue in need thereof a therapeutically effective combination of a membrane sealing surfactant and a cofactor treatment of a cellular energy store and an antioxidant. In some presently contemplated embodiments, a suitable membrane sealing surfactant can comprise a surfactant copolymer (i.e., surfactant copolymer) such as, for example, a poloxamer, a meroxapols, a poloxamine, a PLURADOT™ polyol and combinations thereof. In some presently contemplated embodiments, the cellular energy store comprises a high energy phosphate compound such as, for example, Adenosine Triphosphate (ATP) or phosphocreatine. In some presently contemplated embodiments, one can provide ATP in the form of ATP-MgCl₂ to restore ion balance and energy dependent processes, respectively. In some presently contemplated embodiments, the antioxidant can comprise one or more antioxidants selected from ascorbic acid (ascorbate or Vitamin C), tocopherol (vitamin E), Vitamin A, mannitol, β-carotene, bioflavonoids, flavonoids, flavones, flavonols, proanthocyanidins, selenium, glutathione, N-acetyl cysteine, superoxide dismutase (SOD), lipoic acid, and coenzyme Q-10 (CoQ10) as well as carotenoids such as lycoprene, lutein and polyphenols. Another approach would be to complex the anti-oxidant to the surfactant copolymer which simply deliver of the agent to the damage site.

In one aspect, the methods and compositions disclosed herein provide an ability to seal damaged cell membranes permeabilized by lipid peroxidation and reduce tissue level oxidative damage to cellular proteins.

In another aspect, the invention enables treatment of ischemic events, including cerebral ischemia, and reperfusion injury associated with ischemic events. In an additional embodiment, the invention permits the treatment of ischemic events in a manner that avoids or minimizes the adverse effects associated with conventional treatments, such as reperfusion injury. In another aspect, the invention relates to the administration of therapeutically effective amounts of membrane sealing surfactant, antioxidant, and a cellular energy store prior to the onset of a ischemia or reperfusion; after the onset of ischemia but prior to the onset of reperfusion; or after the onset of both ischemia and reperfusion has occurred.

In another aspect, the invention enables treatment of cell exposed to directly ionizing or indirectly ionizing radiation. In an embodiment in which ionizing radiation has lead to peroxidation of the cellular membrane, administering a therapeutic combination of membrane sealing surfactant and a cofactor treatment consisting of a cellular energy store and an antioxidant increases cell viability relative to cells that receive no treatment or cells in which only the membrane sealing surfactant or the cofactor treatment has been administered.

In another aspect, the invention enables treatment of cells that have suffered permeabilization of the cellular membrane as a result to exposure of extreme thermal conditions such as burns or frostbite.

In another aspect, the invention enables treatment of physiological conditions arising from a breakdown in the barrier function of the cellular membrane. Representative conditions can include cerebral palsy and muscular dystrophy.

In another aspect, a representative advantage of the invention lies in the ability of the therapeutic composition to seal damaged cell membranes permeabilized by lipid peroxidation, combined with reduction of tissue level oxidative damage to cellular protein.

In another aspect, therapeutic combinations of membrane sealing surfactant and a cofactor consisting of antioxidant and a cellular energy store can be provided in pharmaceutically acceptable carriers such as, for example, any and all solvents dispersion media, coatings, antibacterial and antifungal agents, isotonic and absorption delaying agents and the like.

In another aspect of the invention, the membrane sealing surfactant and cofactor treatment can be combined and administered in a single combination with each other or alternatively, can be administered separately from one another or in more than one combination.

In another representative embodiment of the invention, a therapeutic combination of membrane sealing surfactant and a cofactor treatment can be administered orally, rectally, parenterally, such as, for example, intravenously or intramuscularly, or in any combination thereof such that delivery is regional and is provided to tissue in need thereof.

In yet another aspect, the invention also relates to pharmaceutical compositions comprising one or more combinations of therapeutically effective amounts of a membrane sealing surfactant and a cofactor treatment consisting of an antioxidant and a cellular energy store dispersed in pharmaceutically acceptable vehicles.

In another illustrative system, the invention relates to pharmaceutical compositions comprising one or more combinations of therapeutically effective amounts of a membrane sealing surfactant and a cofactor treatment consisting of an antioxidant and a cellular energy store provided separately from one another. In another illustrative system the pharmaceutical compositions can be provided in a single admixture or multi-admixtures with one another.

In one illustrative system, the membrane sealing surfactant comprises poloxamers, meroxapols, poloxamines, PLURADOT™ polyols or combinations thereof.

In another illustrative system, the antioxidant comprises ascorbic acid (Vitamin C, ascorbate), tocopherol (Vitamin E), Vitamin A, mannitol, bioflavonoids, flavonoids, flavones, flavonols, proanthocyanidin, selenium, glutathione, N-acetyl cysteine, superoxide dismutase (SOD), lipoic acid, coenzyme Q-10 (CoQ10), carotenoids such as β-carotene, lycoprene, lutein or polyphenol or combinations thereof.

Representative systems of the invention can be used for the treatment of tissue wherein such treated tissue comprises mammalian tissue. As used throughout the present disclosure, the term “mammal or mammalian” is used herein to comprise all vertebrate mammals, including humans. The terms mammal or mammalian further includes an individual mammal in all stages of development, including embryonic and fetal stages. In an illustrative system, mammals include humans, horses, rodents and canines.

As used throughout the specification and the appended claims, the term “treatment,” in its various grammatical forms, refers to preventing, alleviating, reducing or curing maladies or other adverse conditions.

As used in this specification and the appended claims, the singular forms “a,” “an” and “the” generally mean “at least one,” “one or more” and other plural references, unless the context clearly dictates otherwise. Thus, for example, references to “a membrane sealing surfactant,” “a high energy phosphate compound” and “an antioxidant” include mixtures of one or more membrane sealing surfactants, one or more high energy phosphate compounds, and one or more antioxidants of the type described herein.

### BRIEF DESCRIPTION OF THE DRAWINGS

These, as well as other objects and advantages of this invention, will be more completely understood and appreciated by referring to the following more detailed description of the presently preferred exemplary embodiments of the invention in conjunction with the accompanying drawings, of which:

FIG. 1 is an illustration of representative processes resulting in cellular membrane permabilization.

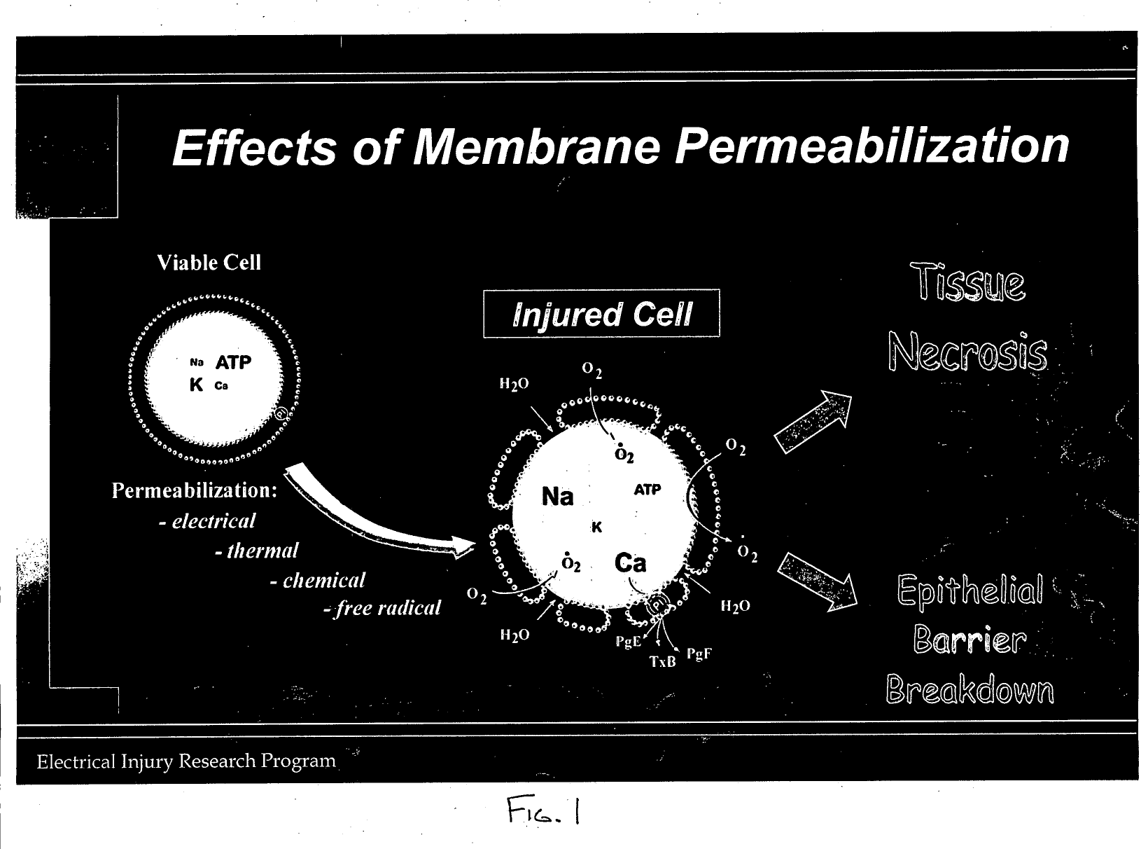

FIG. 2 is an illustration of a representative process wherein ionizing radiation results in cellular membrane permeabilization.

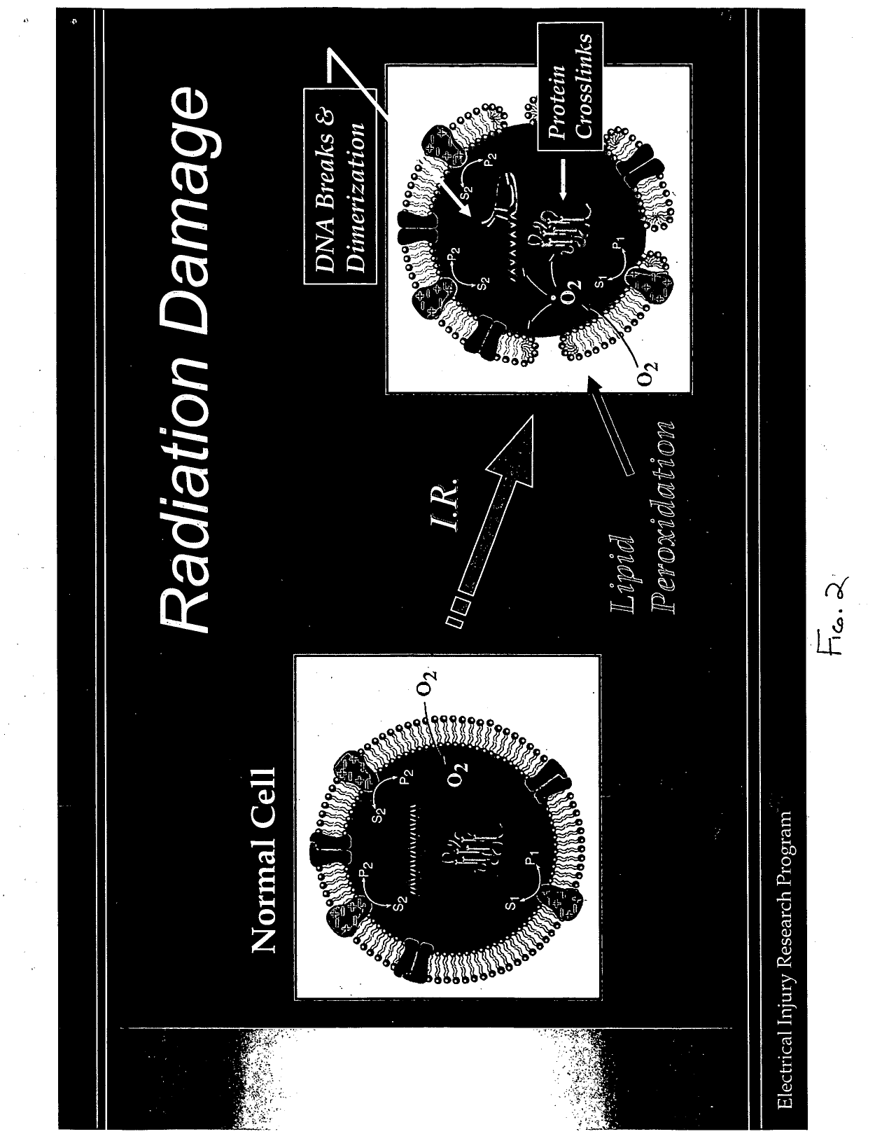

FIG. 3 is an illustration describing the effects of radiation on biomaterials.

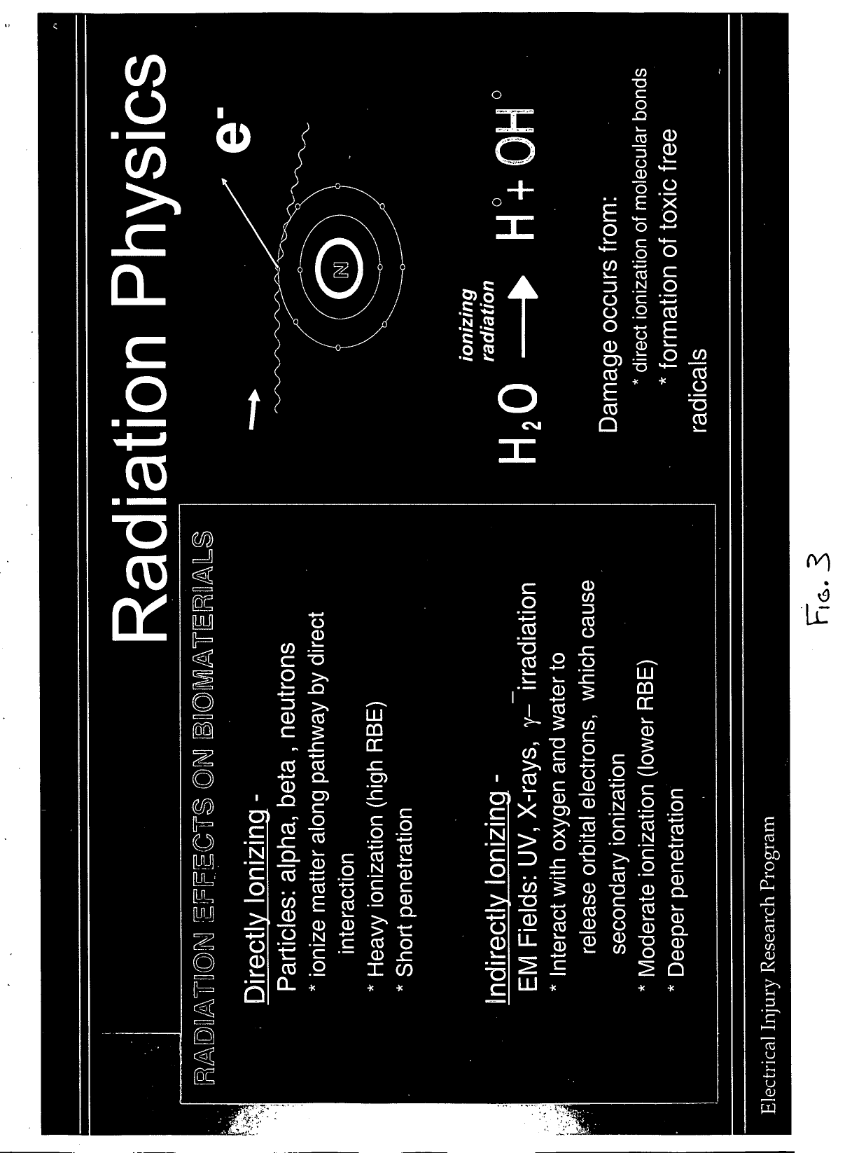

FIG. 4 is an illustration depicting chemical structures of representative polaxamer based surfactants.

FIG. 5 is an illustration depicting the effects of a representative poloxamer applied to a permeabilized cell membrane.

FIG. 6 is an illustration illustrating an experimental protocol for testing the effects of a therapeutic composition of the present invention on radiation exposed mammalian cells.

FIG. 7 is an illustration of mammalian cell viability following exposure to varying levels of radiation.

FIG. 8 is an illustration of cell viability results for mammalian cells treated with different surfactants following exposure to 40 Gy of radiation.

FIG. 9 is an illustration of cell viability results for mammalian cells treated with a variety of treatments at a time 18 hours subsequent to 40 Gy of radiation exposure.

FIG. 10 is an illustration of cell viability results for mammalian cells treated with a variety of treatments at a time 48 hours subsequent to 40 Gy of radiation exposure.

### DESCRIPTION OF ILLUSTRATIVE EMBODIMENTS

The present inventors discovered that cellular necrosis could be reduced, and in certain circumstances prevented, if the barrier characteristics of a peroxidized cellular membrane was restored in combination with therapy to reduce tissue level oxidation and restore cellular energy levels. To affect this goal, the permeability of damaged cellular membranes is reestablished—effectively “sealing” the injured membranes. To facilitate rapid tissue recovery, cellular energy levels can be reestablished through addition of a cellular energy source such as, for example, phospocreatine, adenosine diphosphate and adensine triphosphate (ATP) in the form of MgCl₂-ATP which, serves a dual benefit of improving the cellular ion balance and an antioxidant eliminating the generation of Reactive Oxygen intermediates and enhancing metabolism of free radicals. Thus, in one embodiment, a therapeutic composition, comprises a therapeutic combination of a membrane sealing surfactant and a cofactor treatment consisting of an antioxidant and a cellular energy store. Such multimode combination therapy can be useful in treating mammalian cells experiencing cellular membrane injury resulting from exposure to events such as colic, acute myocardial infarction, ischemia/reperfusion injury, cerebral palsy, muscular dystrophy, stroke, spinal cord injury, head injury, organ transplantation, inflammatory bowel conditions, cancer, severe infectious disease, necrotizing endocolitis, bacterial translocation, conditions characterized by exposure to ionizing radiation (IR), conditions characterized by exposure to chemical oxidants which produce excess reactive oxygen species and certain other surgical procedures.

For example, ischemia/reperfusion (I/R) injury plays an important role in a wide variety of clinical situations. Most therapies used to treat or study I/R injury function primarily by attempting to interrupt damaging enzymatic pathways by either (i) preventing the generation of oxygen free radicals and/or (ii) enhancing the capacity of a tissue to metabolize oxygen free radicals. Certainly, other pathways and components are likely to be activated and/or maintained in I/R. Thus, the present inventors have proposed to combine therapeutic measures that effect sealing of damaged cell membranes with the reduction of the oxygen free radicals for treatment and/or prevention of I/R injury. Thus, in an illustrative embodiment, the invention provides methods and compositions for the treatment and prevention of I/R injury.

When ischemia occurs in a tissue, membrane depolarization occurs followed by increased cellular permeabilization. Increased permeability rapidly results in the following events: disruption of calcium ion and amino acid balance, sodium ion imbalance, cellular swelling and neurotransmitter imbalance. Cellular damage is further enhanced by the inhibition of protein synthesis and depletion of intracellular energy stores caused by the ischemia. With the onset of reperfusion, the increased intracellular calcium activates many damaging pathways which cause further damage, including intravascular thrombosis and tissue inflammation.

An advantage of the invention is that by sealing the damaged cell membranes, the chemicals that activate certain damaging pathways are no longer released into the interstitial tissues by the damaged cells. Thus, once the chemicals have been metabolized, these damaging pathways are no longer stimulated and further damage is obviated.

Current therapies which prevent the generation of oxygen free radicals or enhance metabolism of oxygen free radicals do not block the initial steps in the enzymatic pathways that they target. Because the initial steps are not blocked, other responses are stimulated and tissue damage is not entirely prevented. As stated above, the inventors discovered that sealing cell membranes will limit the amount of chemicals that are available to cause additional immune responses and increase tissue damage. However, it is advantageous to provide agents that reduce oxidation in order to further protect tissues.

It is also understood that more than one membrane sealing surfactant, antioxidant or cellular energy store may be combined in the invention. For example, it may be desirable to use a rapid release formulation of one cellular energy store agent in combination with an extended release formulation of the same or even a different cellular energy store agent.

A. Membrane Sealing and Sealing Surfactants

Membrane sealing surfactants, also referred to as surfactant copolymers, or block polymer nonionic surfactants, are surfactant agents prepared by linking two or more biopolymers into a single multiblock copolymer with at least one block being hydrophobic. A membrane sealing surfactant having a combination of a hydrophilic polymer and hydrophobic polymer will generally be suitable for use with the present invention if the molecular size is large enough to prevent affecting normal proteins or membranes. In one common embodiment, the sequential addition of two or more aklelene oxides to a low molecular weight water soluble organic compound containing one or more active hydrogen atoms. These latter compounds are described in U.S. Pat. No. 5,470,568, which is herein incorporated by reference.

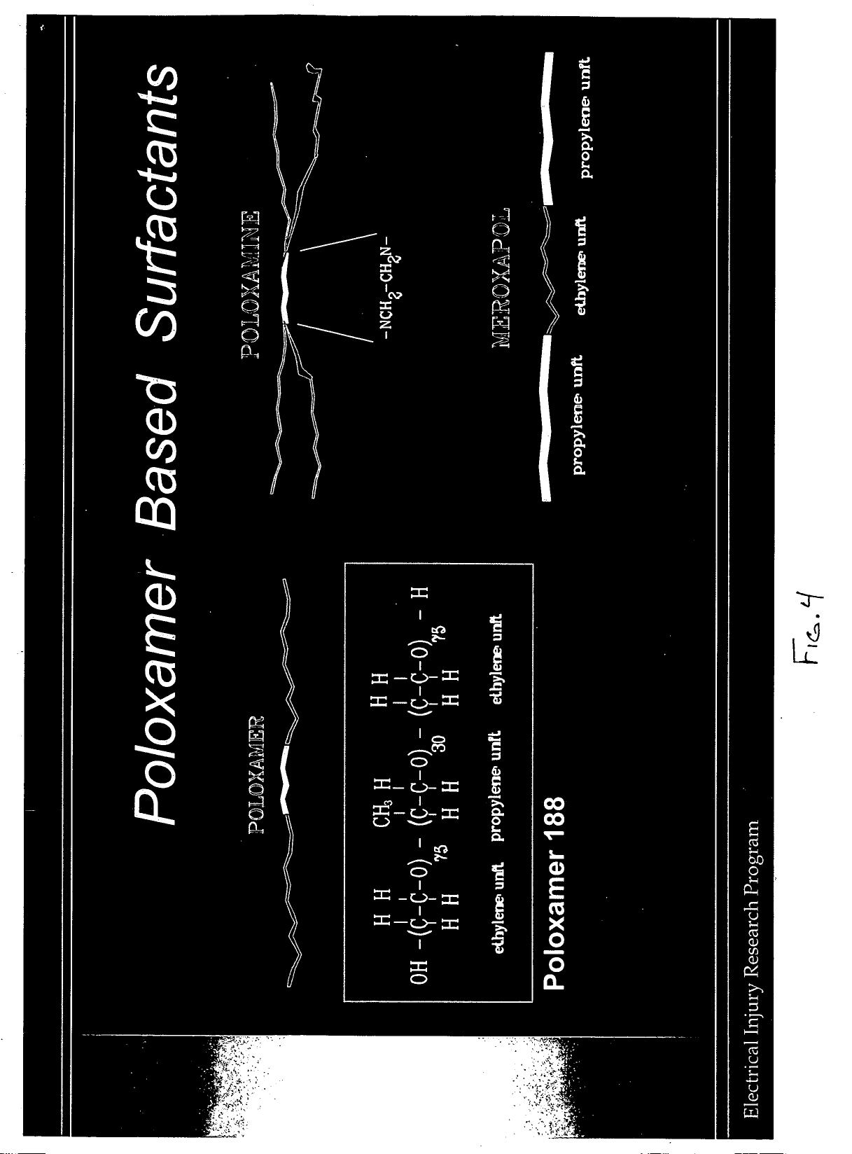

Representative groups of membrane sealing surfactants contemplated for use with regard to the present invention include the poloxamers, the meroxapols, the poloxamines and the PLURADOT™ polyols, all commercially available from suppliers such as the BASF Corporation. There is a good deal of intergroup variation with respect to the polymers' synthesis, although in all syntheses the oxyalkylation steps are carried out in the presence of an alkaline catalyst, generally sodium or potassium hydroxide. The alkaline catalyst is then neutralized and typically removed from the final product. Structures for representative membrane sealing surfactants including a poloxamer, poloxamine and meroxapol are as illustrated in FIG. 4. Almost any combination of hydrophilic polymer and hydrophobic polymer will work if the molecular size is large enough to prevent affecting normal proteins or membranes.

Poloxamer 188 (P188) available from BASF Corp. of Parsippany, N.J., has been shown to block the adhesion of fibrinogen to hydrophobic surfaces and the subsequent adhesion of platelets and red blood cells. It is an FDA-approved surfactant in the synthetic blood replacement fulsol (Check and Hunter, 1988 and also, U.S. Pat. Nos. 4,879,109; 4,897,263; and 4,937,070, incorporated herein by reference). The poloxamers are synthesized by the sequential addition of propylene oxide, followed by ethylene oxide, to propylene glycol, which in the case of the poloxamers constitutes the water-soluble organic component of the polymer. The inner polyoxy-propylene glycol is the hydrophobic portion of the poloxamer. This is due to the fact that this group changes from a water-soluble to a water-insoluble polymer as the molecular weight goes above 750 Daltons. Adding ethylene oxide in the final step makes the molecule water-soluble.

In one embodiment of the invention, the use of a poloxamer with a molecular weight of at least 2,000 and not more than 20,000 Daltons is useful. This molecular weight range is useful in maintaining the appropriate solubility of the poloxamer in water while minimizing or eliminating any potential toxicity. Furthermore, the poloxamer's hydrophobic group should have a molecular weight of approximately 45-95% by weight of the poloxamer. More preferably, the hydrophobic group should have a molecular weight of 1,750-3,500 Daltons, and the hydrophilic groups should constitute 50-90% by weight of the molecule. The relative amounts of hydrophile and the molecular weight of the hydrophobe are critical to several of the poloxamer's properties, including its solubility in water and its interactions with hydrophobic groups, and the illustrative ranges provided in the present invention provide the maximum effectiveness currently known while minimizing or eliminating toxicity.

When the order of addition of the alkylene oxides is reversed, the meroxapol series is produced. In this series, ethylene glycol is the initiator, and as opposed to the poloxamers, which are terminated by two primary hydroxyl groups, the meroxapols have secondary hydroxyl groups at the ends and the hydrophobe is split in two, each half on the outside of the surfactant.

The poloxamines are prepared from an ethylene diamine initiator. They are synthesized using the same sequential order of addition of alkylene oxides as used to synthesize the poloxamers. Structurally, the poloxamines differ from the other polymers in that they have four alkylene oxide chains, rather than two, since four active hydrogens are present in the initiator. They also differ from the other surfactants in that they contain two tertiary nitrogen atoms, at least one of which is capable of forming a quaternary salt. The poloxamines are also terminated by primary hydroxyl groups.

The PLURADOT™ polyols (a quad-block surfactant composed of a block copolymer of trimethylolpropane attached to three blocks of polyoxyethylene can be prepared from a low molecular weight trifunctional alcohol, such as glycerine or trimethylpropane, which is oxyalkylated initially with a blend of propylene and ethylene oxides, but primarily with propylene oxide, to form the hydrophobe. This is followed by oxyalkylating with a blend of ethylene and propylene oxiles, but primarily ethylene oxide, to form the hydrophile. This group of surfactants has three chains, one more than the poloxamer and meroxapol series, but one less than the poloxamine polymers.

The hydrophilic and hydrophobic chains of the surfactant copolymers each have unique properties which contribute to the substances' biological activities. With regard to poloxamers in particular, the longer the hydrophilic polyoxyethylene chains are, the more water the molecule can bind. As these flexible chains become strongly hydrated they become relatively incompressible and form a barrier to hydrophobic surfaces approaching one another. The hydrophobic component of the poloxamers is typically large, weak and flexible.

In any of the surfactant copolymer series, as the percent of ethylene oxide increases, or the molecular weight of the hydrophobe decreases, the solubility of the molecule in water increases. Of the four groups of copolymers only the meroxapol polymers exhibit any solubility in mineral oil. The higher the hydrophobic molecular weights, the less soluble the copolymer will be in an organic solvent, and the same is true for those polymers with higher ethylene oxide propylene oxide concentration. The molecular weight of the hydrophobe will also affect the wetting time of any one species, and the ethylene oxide/propylene oxide ratio of the molecule will influence the foaming properties of that copolymer. A copolymer's emulsification properties may correlate with hydrophobe molecular weights, and the toxicity decreases as the ethylene oxide/propylene oxide ratio increases and as the molecular weight of the hydrophobe increases.

The four groups of presently contemplated membrane sealing surfactants are alike in that they derive their solubility in water from hydrogen bond formation between the many oxygen atoms on the copolymer and protons in the water. As the temperature of a solution containing a nonionic surfactant is raised, the hydrogen bonds are broken and the copolymer clouds out of solution. For example, for poloxamers, the 1% cloud point ranges from a low of 14° C. to a high of 100° C., the latter figure being the cloud point for the most hydrophilic polymers. The poloxamines are similar structurally to the poloxamers, and their cloud point range is similarly wide. On the other hand, the meroxapols have a much narrower cloud point range, and the PLURADOT™ polymers have the lowest maximum cloud point, primarily due to their lower ethylene oxide content.

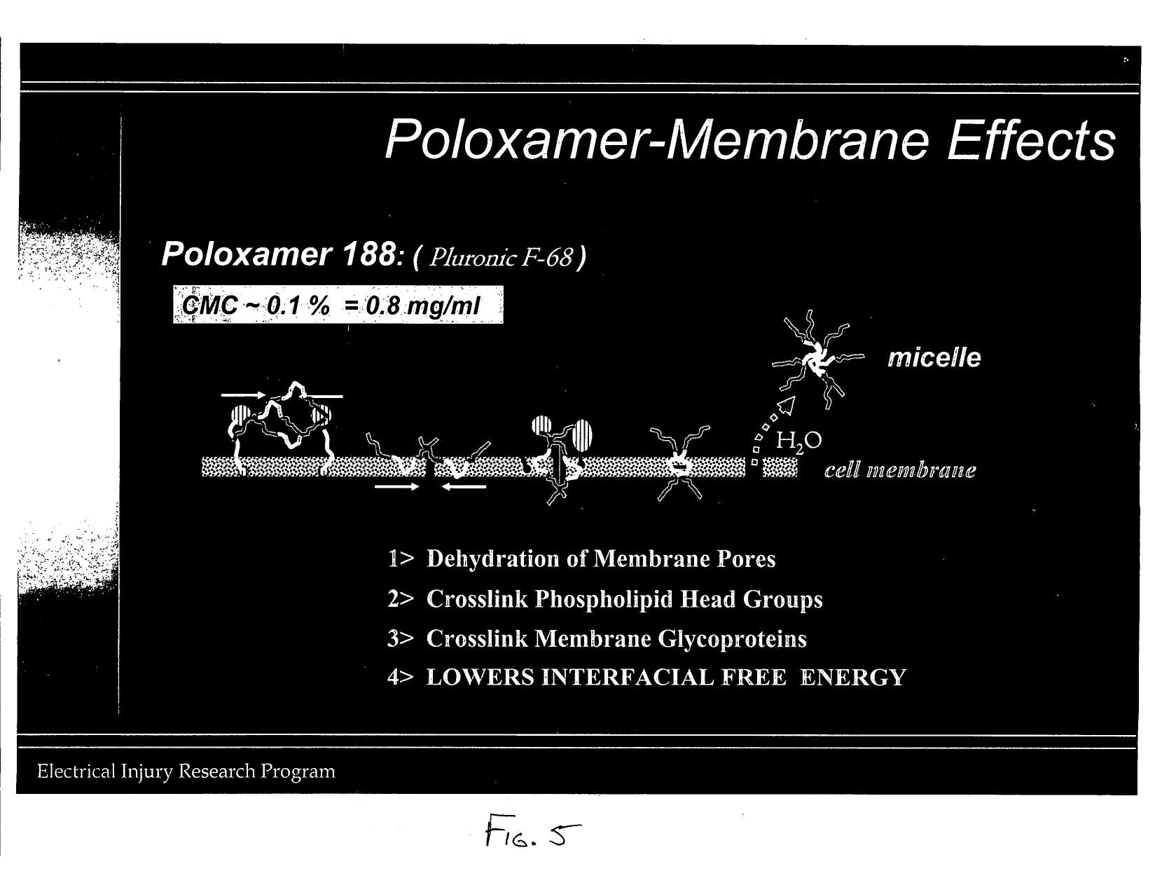

Surfactant copolymers are capable of preventing or minimizing cell membrane permeabilization and repairing permeabilized membrane as illustrated in FIG. 5 and as described in U.S. Pat. No. 5,605,687 and U.S. Patent Pubs. US2003/0118545A1 and US2005/0069520A1, which are herein incorporated by reference. It has been suggested that the hydrophobic central domain of the polymer may bind to the hydrophobic portion of the lipid bilayer when those groups are exposed following removal of the external layer of the membrane. The manner in which the poloxamer is folded when this binding occurs has been postulated to assist in the restoration of a nonadhesive cell surface. Poloxamers are surprisingly capable not merely of restoring a nonadhesive surface, but actually of repairing or potentiating the repair of complete permeations of the entire membrane bilayer.

B. Cofactor Treatment

As the membrane sealing surfactant reestablishes and seals the damaged cellular membrane, the ion balances and cellular energy stores of the damaged cell can be replenished while simultaneously preventing further attack to the cellular membrane from free radicals and/or Reactive Oxygen Intermediates (ROI). In combination with the afore discussed membrane sealing surfactant, a cofactor treatment consisting of an antioxidant and a cellular energy store is administered as part of the therapeutic composition to yield a desirable synergistic effect.

i. Antioxidants

A wide variety of antioxidants are contemplated as being useful for the treatment of free radical mediated injury of the cellular membrane. One or more antioxidants may be used in combination with each other along with a suitable membrane sealing surfactants and a cellular energy store. Compositions having antioxidant properties and contemplated as being useful in the invention have been previously described in U.S. Pat. Nos. 5,725,839; 5,696,109; 5,691,360; 5,683,982; 5,659,055; 5,659,049; 5,648,377; 5,646,149; 5,643,943 and 5,623,052, all of which are incorporated herein by reference.

Illustrative compositions having antioxidant properties which are contemplated as being useful in the invention include ascorbic acid (ascorbate or Vitamin C), tocopherol (vitamin E), Vitamin A, mannitol, β-carotene, bioflavonoids, flavonoids, flavones, flavonols, proanthocyanidins, selenium, glutathione, N-acetyl cysteine (NAC), superoxide dismutase (SOD), lipoic acid, and coenzyme Q-10 (CoQ10). Carotenoids such as lycoprene, lutein and polyphenols are also contemplated as being useful.

In general, antioxidants useful in the invention either improve brain parenchymal penetration, suppress reduction of mitochondrial function during ischemia and promote restoration of such during reperfusion, significantly suppress reduction of glutathione levels in liver tissue, rapidly restore liver tissue ATP levels during reperfusion after such levels have been reduced during ischemia, significantly suppress elevation of lipid peroxidation following reperfusion and/or significantly suppress elevation of the concentration of adenine nucleotides in the blood stream.

It is understood that certain antioxidants may be more desirable for use before ischemia, after ischemia but prior to reperfusion or after both ischemia and reperfusion have occurred. It is also understood that certain antioxidants when combined may have a greater than additive effect.

Recommended dose ranges and individualization of dosage of antioxidants approved for clinical use in the United States are found in the Physicians' Desk Reference, 52^{nd} Ed., 1998, incorporated herein by reference.

ii. Cellular Energy Store

Many cellular processes require stored energy. When the cellular membrane has been damaged and permeabilized, the normal barrier function of the cell membrane is eliminated and the stored cellular energy is lost and/or depleted as the cell attempts to restore the ion balance. As the cell energy is depleted, levels of calcium rise in the cell, which can lead to the formation of damaging free radicals as well as turning on cell death signals.

The most common form of stored cellular energy are high energy phosphate compounds. High energy phosphate compounds generally comprise pyrophosphate bonds and acid anhydride linkages formed by taking phosphoric acid derivatives and dehydrating them. High energy phosphate compounds react with a variety of cellular processes to provide the energy allowing the processes to run, control the process by coupling the process to a particular nucleoside and by driving the process from a reversible process to an irreversible process. Representative high energy compounds contemplated as being useful in combination with membrane sealing surfactants and antioxidants as previously described, include Adenosine Triphosphate (ATP), Adenosine Diphosphate (ADP) and phosphocreatine.

ATP is the high energy phosphate compound found generally in all cells. ATP comprises an ordered carbon backbone having a triphosphate (three phosphorous groups connected by oxygen atom). Each phosphorous atom further includes a side oxygen atom. Removing one of the phosphate groups from ATP releases stored energy for use within the various cellular processes and consequently results in the formation of Adenosine Diphosphate (ADP). ADP can be subsequently converted back to ATP through the oxidation of glucose in the Krebs cycle such that stored energy in the form of ATP is again available to the cell.

One illustrative cellular energy source contemplated as being useful in the cofactor treatment of the invention includes ATP available as MgCl₂-ATP. MgCl₂-ATP can be beneficial not only for its ability to replenish cellular energy stores but also by increasing levels of MgCl₂, the cellular ion balance is improved.

Creatine-Phosphate is another high-energy compound which can be used.

C. Pharmaceutical Compositions

Aqueous compositions of the present invention comprise an effective amount of the previously discussed membrane sealing surfactants and cofactor treatment dissolved or dispersed in a pharmaceutically acceptable carrier or aqueous medium. The phrases “pharmaceutically or pharmacologically acceptable” refer to molecular entities and compositions that do not produce an adverse, allergic or other untoward reaction when administered to an animal, or a human, as appropriate.

As used herein, “pharmaceutically acceptable carrier” includes any and all solvents dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. For human administration, preparations should meet sterility, pyrogenicity, general safety and purity standards as required by FDA Office of Biologics standards.

The biological material should be extensively dialyzed to remove undesired small molecular weight molecules and/or lyophilized for more ready formulation into a desired vehicle, where appropriate. The active compounds will then generally be formulated for parenteral administration, e.g., formulated for injection via the intravenous, intramuscular, subcutaneous, intralesional, or even intraperitoneal routes. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringabilty exists. It must be stable under the conditions of manufacture and storage and must be preserved against the contaminating action of microorganisms, such as bacteria and fungi.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimersosal, and the like. In many cases, it will be preferable to include isotonic agents, for example, sugars or sodium chloride. Prolonged absorption of the injectable compositions can be brought about by the use in the compositions of agents delaying absorption, for example, aluminum monosterate and gelatin.

Sterile injectable solutions are prepared by incorporating the membrane sealing surfactants and cofactor treatment in the required amount in the appropriate solvent followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15^{th} Ed., pages 1035-1038 and 1570-1580). Some variation in dosage will necessarily occur depending on the condition of the subject being treated. The person responsible for administration will, in any event, determine the appropriate dose for the individual subject.

The agents may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds formulated for parenteral administration, such as intravenous or intramuscular injection, other pharmaceutically acceptable forms include e.g., tablets or other solids for oral administration; liposomal formulations; time release capsules; and any other form currently used, including creams.

Additional formulations which are suitable for other modes of administration include suppositories. For suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders.

In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule it may contain, in addition to materials of the above type, a liquid carrier. Various other materials may be present as coatings or to otherwise modify the physical form of the dosage unit. For instance, tablets, pills or capsules may be coated with shellac, sugar or both. A syrup or elixir may contain the nonactive compounds sucrose as a sweetening agent, methyl and propylparabens as preservatives, a dye, and flavoring such as cherry or orange flavor.

D. Dosage and Administration

The skilled artisan will recognize that certain combinations of drugs are recommended only for certain conditions or that in some cases certain drugs or combinations of drugs are contraindicated. Further, individual patients may respond better to one combination of drugs in one set of circumstances and in another set of circumstances respond more favorably to a different drug combination. Contemplated routes include oral, topical, vaginal, rectal, ophthalmic, intravenous, intramuscular, subcutaneous, intralesional, or even intraperitoneal routes. Also treatment of open wounds and surgical sites are within the scope of the inventions.

Factors that are well known to influence patient response to drug therapy include, but are not limited to, species, age, weight, gender, health, pregnancy, addictions, allergies, ethnic origin, prior medical conditions, current medical condition and length of treatment. Thus, the skilled artisan will be well acquainted with the need to individualize dosage(s) and the route(s) of administration to each patient.

The skilled artisan will also consider the condition that is to be treated prior to selecting the appropriate combination of drugs. For example, an admixture that is appropriate for the pretreatment of a patient prior to surgery, and the subsequent ischemia associated with the surgery, may not be the desired combination for a patient suffering from acute myocardial infarction or stroke.

The skilled artisan will further recognize that both the route of administration and the form of administration can significantly influence the dosage. For example, the dosage used with the oral administration of a drug in an extended release form may be more than ten-fold greater than the dosage of the same drug administered intravenously.

Thus it is recognized that in the practice of the invention a wide variety of dosages and routes of administration may be useful.

For example, a therapeutic composition of the present invention could comprise a therapeutically effective dose of membrane sealing surfactant, such as, for example, a poloxamer, a meroxapol, a poloxamines, a PLURADOT™ polyols and combinations thereof in an amount ranging from about 0.01 mg/ml of blood volume to about 5.0 mg/ml blood volume, and preferably from about 0.1 mg/ml of blood volume to about 5.0 mg/ml of blood volume. A person of ordinary skill in the art will realize that additional ranges of membrane sealing surfactant dosages are contemplated and are within the present disclosure.

In addition, representative therapeutic compositions comprise a dose of antioxidant, such as, for example, ascorbic acid (ascorbate or Vitamin C), tocopherol (vitamin E), Vitamin A, mannitol, β-carotene, bioflavonoids, flavonoids, flavones, flavonols, proanthocyanidins, selenium, glutathione, N-acetyl cysteine (NAC), superoxide dismutase (SOD), lipoic acid, coenzyme Q-10 (CoQ10), carotenoids such as lycoprene, lutein and polyphenols and combinations thereof, at dose levels appropriate for each antioxidant. For example, representative therapeutically effective dose ranges can comprise:

| Antioxidant | Dose Level (mg) |
|---|---|
| Vitamin C | 100-1,500 |
| CoQ10 | 5-50 |
| NAC | 25-1,000 |

A person of ordinary skill in the art will realize that additional ranges of antioxidant dosages are contemplated and therapeutically effective amounts of antioxidants can be selected based on the efficacy of the particular compound as well as safe ranges of the compounds.

Representative therapeutic compositions further comprise cellular energy sources, such as, for example, Adenosine Triphosphate (ATP), Adenosine Diphosphate (ADP) and phosphocreatine, at therapeutically effective dose levels from about 0.1% to about 15% w/v (component weight to volume of composition). For example, a dose of ATP can be provided through the inclusion of MgCl₂-ATP at a dose of about 0.1% w/v or phosphocreatine at a dose of about 10% w/v. A person of ordinary skill in the art will realize that additional ranges of cellular energy source dosages are contemplated and are within the present disclosure.

E. Illustrative Example

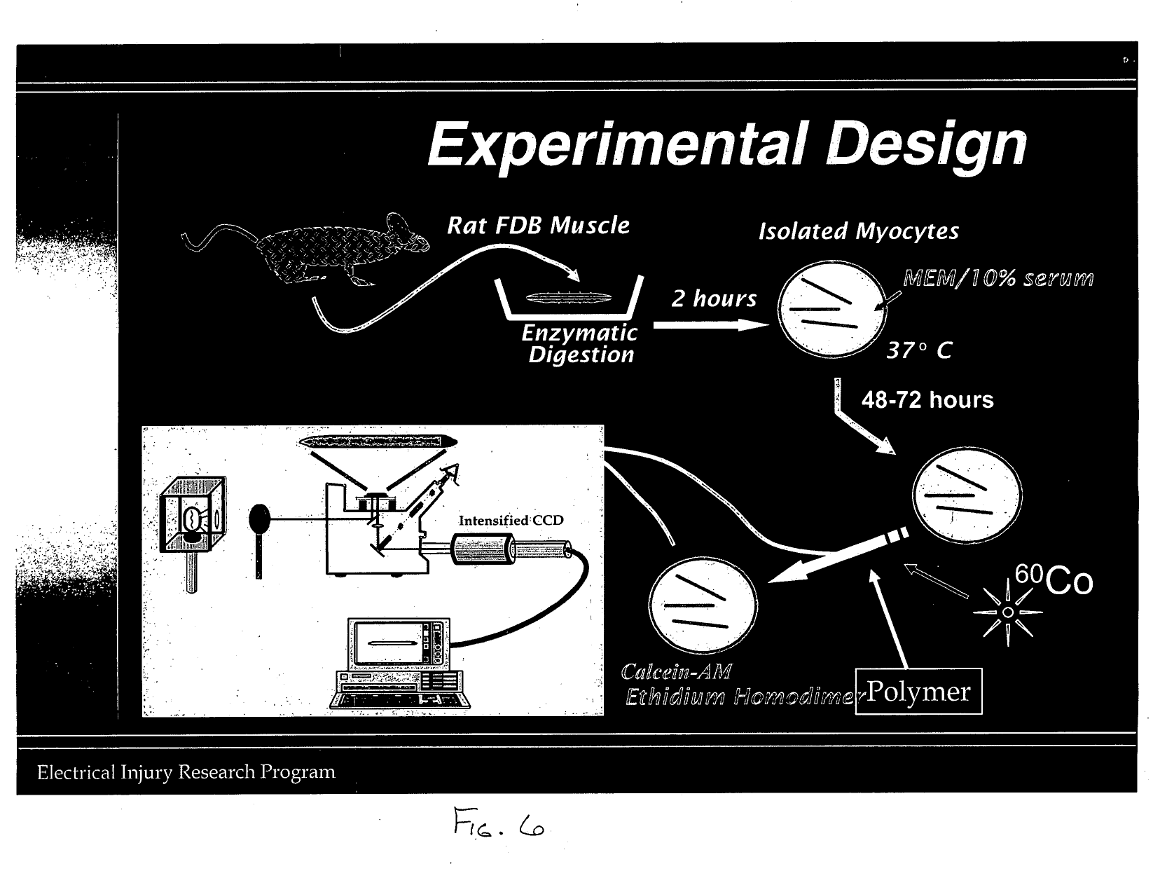

In order to illustrate the benefits and advantages of the therapeutic composition of the present invention, mammalian rat cells were harvested and exposed to directly ionizing radiation resulting in peroxidation of the cellular membrane. To facilitate testing and experimentation, radiation exposure was utilized to achieve cellular membrane peroxidation, though it is to be understood that similar peroxidation of the cellular membrane is achieved through exposure to a variety of alternative systemic and external events such as, for example, colic, acute myocardial infarction, ischemia/reperfusion injury, cerebral palsy, muscular dystrophy, stroke, spinal cord injury, head injury, organ transplantation, necrotizing endocolitis, bacterial translocation, and conditions characterized by exposure to chemical oxidants which produce excess reactive oxygen species, all of which are known to lead to cellular membrane peroxidation and consequently, cell death. The testing protocol is as described below and summarized in FIG. 6.

i. Materials and Methods

Flexor digitorum brevis skeletal muscle cells were harvested from 4-week-old female Spague-Dawley rats obtained from Harlen-Sprague-Dawley Inc., Indianapolis, Ind. at the University of Chicago Carlson Animal Facility. The muscle tissue was harvested within 20 minutes following sacrifice of the rats by asphyxiation. The samples were then soaked in 18-20 h in 0.3% collagenase type III and 0.35% trypsin (Worthington Biochemical Corp., New Jersey) in a phosphate-buffered saline solution containing calcium and a pH buffer N-2-hydroxyethylpiperazine-n-2-ethansulfonic acid. Cells were then incubated for 32 minutes at 37° C. in order to separate them. Cells were then washed and separated by trituration and distributed onto tissue culture dishes (Falcon, Cambridge, Mass.), 250-300 at a time. Cells were allowed to recover from tituration, remaining untouched for 3 days at 37° C. and 95% relative humidity in Minimum Essential Medium (Gibco BRL, Grand Island, N.Y.) inside a water jacketed incubator (ThermoForma Scientific model 3326, Marietta, Ohio). The medium was supplemented with 25 mM HEPES, 10% Nu-Serum (Collaborative Biomedical products of Becton Dickinson, Bedford, Mass.), 50 U/ml penicillin, and 100 mcg/ml Streptomycin (Gibco BRL).

In preparation for post IR viability testing, an initial viability measurement was done the third day after cell harvesting. Ethidium homodimer-1 (EH), dissolved in 1:4 DMSO/water and calcien-AM (molecular probes, Oregon) dissolved in dry DMSO were added from stock solutions to stain the cells at final concentrations of 10 and 3.3 mcM, respectively. EH fluoresces with a red color (λex=528 nm; λem=617 nm) after binding to DNA within the cell. Entrance of this molecule, Mr=856.77, into enter the cell indicates significant cell membrane destabilization and cell death. Calcein-AM flourescence requires ATP-dependent cleavage which occurs in the cell's cytosol. Its green fluorescence (λex=494, λem=517 nm) indicates that the cell maintains both metabolic capabilities as well as a stable membrane. Cells demonstrating any accumulation of EH were deemed inviable even if green fluorescence was still appreciated. Fifteen minutes after the dye was added to these dishes, their fluorescence was assessed using a Nikon Diophot inverted microscope with fluorescent optics.

Two or three dishes from each cell batch were tested. If 70% of the cells tested were viable, they were considered healthy and suitable for the IR experiment. The dishes were then divided into various batches: cell batches for non-IR sham-exposed controls; cell batches for IR exposed non-treated controls; cell batches for IR exposed and treated with 1 mM P188; cell batches for IR exposed and treated with 1 mM Dextran; cell batches for IR exposed and treated with 2 mM P188; cell batches for IR exposed cells treated with cofactor treatment comprising 10 mM N-Ac-Cysteine and 0.1 mM MgCl₂-ATP; cell batches for IR exposed and treated with the therapeutic composition of the invention comprising 1 mM P188 and cofactor treatment (10 mM N-Ac-Cysteine and 0.1 mM MgCl₂-ATP); cell batches for IR exposed and treated with 1 mM p188+10 mM NAC+0.1 mM Mg-ATP; and two dishes irradiated and treated with 1 mM P188.

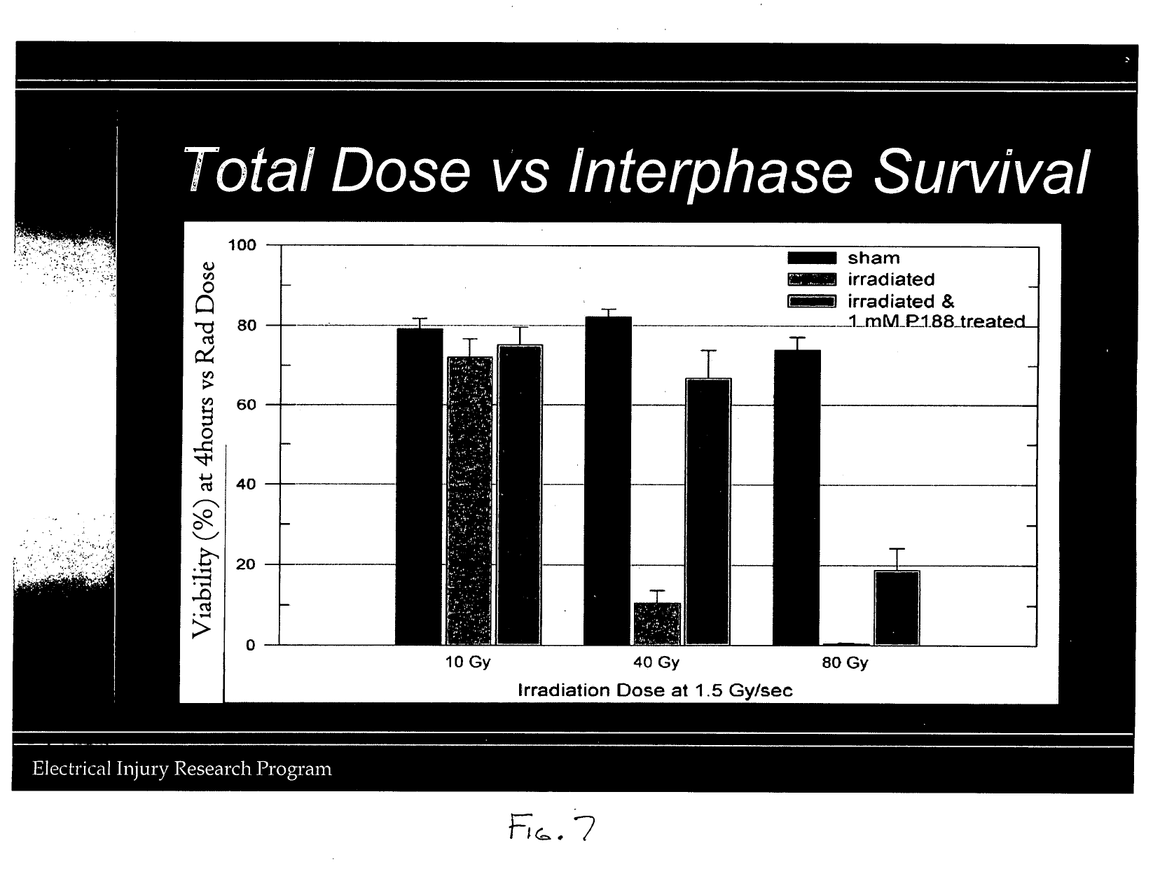

When transported to the IR chamber, the cells were placed on top of a 37° C. heating pad, and then inside of an insulated box in order to minimize temperature variation between the samples. The sham-exposed samples were transported along with the IR-treated samples in order to be subjected to the same temperatures and motion stresses. A decrease in viability of sham-exposed cells by more than 20% was interpreted as defective and was discarded. Initially, cell batches including the non-IR exposed sham cells, the IR exposed non-treated cells and the IR exposed cells treated with 1 mM P188 were exposed to 60Co gamma radiation provided by a gammacell 220 (AECL, Chalk River, Ontario, Canada). Cell viability results at various IR doses (10 Gy, 40 Gy, 80 GY) is illustrated in FIG. 7. The experiments demonstrated little difference in the viability of P188 treated cells a compared to the IR exposed non-treated cells at 10 Gy. At a dose level of 80 Gy, cell viability was improved for the P188 treated cells, but not markedly over the IR exposed non-treated cells. Treatment of cells with P188 effected the greatest improvement in viability for cells treated with P188 vs. no treatment at a dose level of 40 Gy. Thus, a radiation dose level of 40 Gy was chosen for the remaining experiments. Exposure time was calculated from a dose-rate calibration table furnished by the University of Chicago Laboratory for Radiation and Oncology Research. Dishes exposed to radiation were placed into a Gammacell unit for the time necessary to receive the correct dose.

Following irradiation of the remaining cell batches at an IR dose level of 40 Gy, all the cell batches were returned to the tissue culture lab where various treatments were added to the irradiated dishes to determine the effects of Dextran treatment versus P188 treatment, 18 hour cell viability data and 48 hour cell viability data. Sham-exposed dishes as well as the remaining IR exposed dishes received additional media culture equivalent to the amount added to the dishes receiving the polymer cocktails.

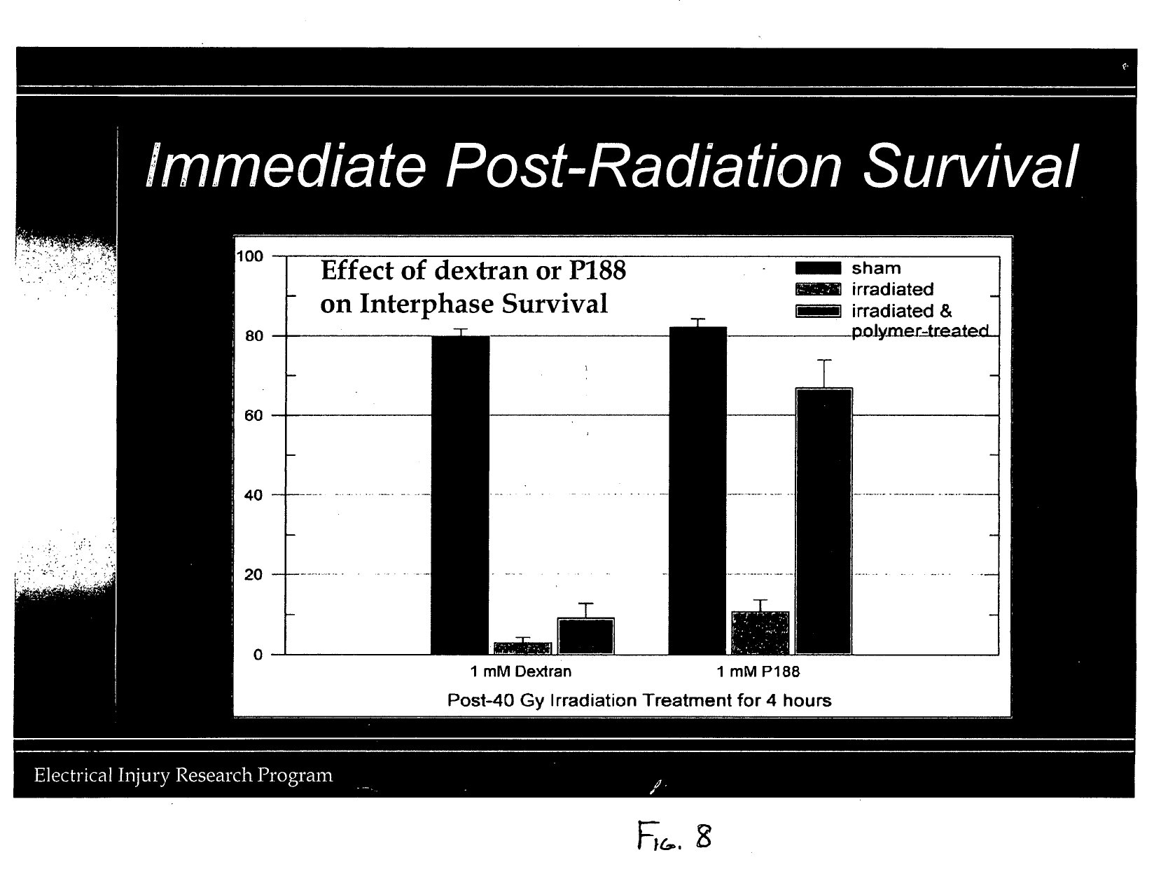

As illustrated in FIG. 8, cell viability was determined for polymer treatment of IR exposed cells using Dextran or P188. While treatment of 1 mM of Dextran offered a slight improvement versus no treatment of IR exposed cells with respect to cell viability, treatment of IR exposed cells with 1 mM of P188 offered substantial improvement on cell viability as compared to no treatment of IR exposed cells.

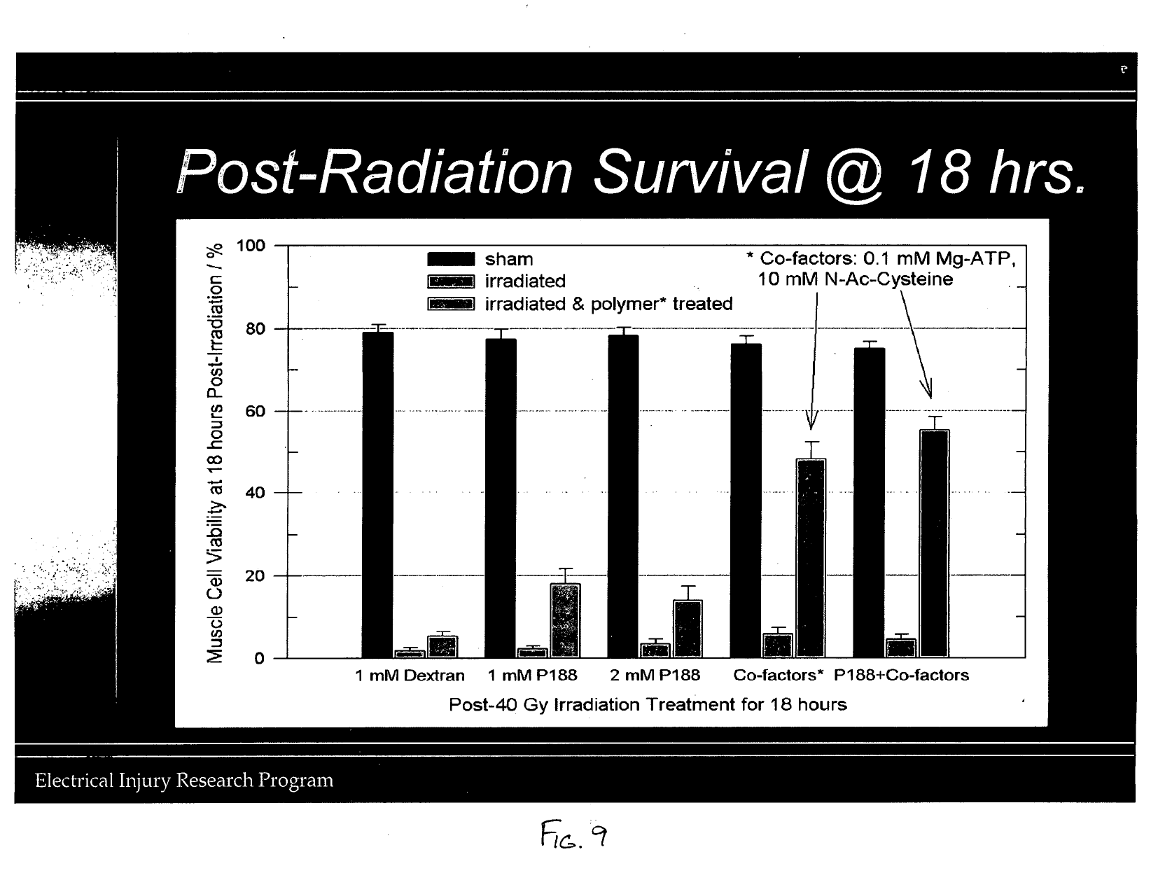

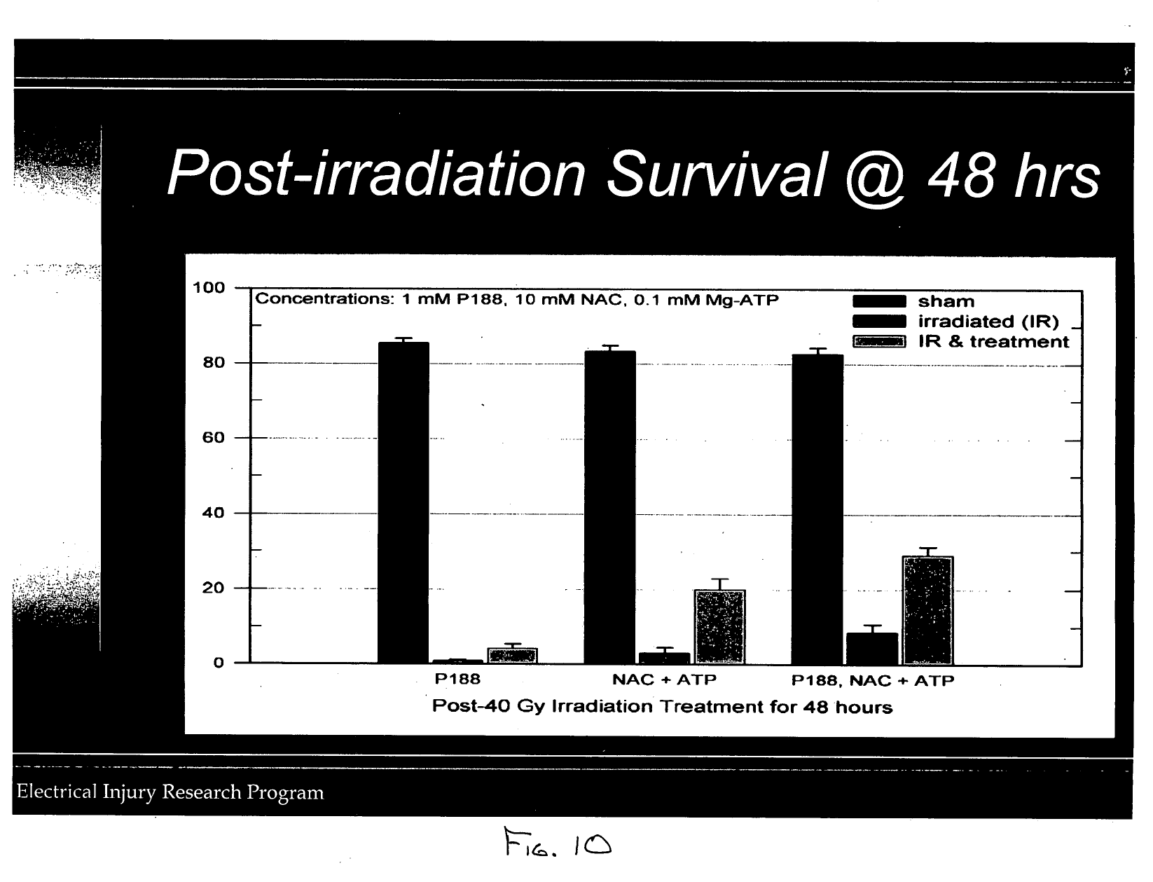

Fluorescent dye was added at 18 and 48 hours post IR exposure to the cell batches in order to observe survival in the same manner used for initial viability testing. The viability of cells at 18 and 48 hours of testing is illustrated in FIGS. 9 and 10, and were determined as the percentage of cells exhibiting calcien fluorescence alone. Our analysis considered the mean percentage viability for the multiple samples done for each testing parameter (Sham IR non-exposed, IR exposed untreated, IR exposed and treated with 1 mM Dextran, IR exposed and treated with 1 mM P188, IR exposed and treated with 2 mM P188, IR exposed and treated with cofactor treatment (0.1 mM MgCl₂ and 10 mM NAC), IR exposed and treated with a therapeutic composition of 0.1 mM P188+cofactor treatment (0.1 mM MgCl₂ and 10 mM NAC), at 18 hours post-IR exposure. In a similar manner, the analysis considered the mean percentage viability for the multiple samples done for each testing parameter (Sham IR non-exposed, IR exposed untreated, IR exposed and treated with 1 mM P188, IR exposed and treated with cofactor treatment (0.1 mM MgCl₂ and 10 mM NAC), IR exposed and treated with a therapeutic composition of 0.1 mM P188+cofactor treatment (0.1 mM MgCl₂ and 10 mM NAC), at 48 hours post-IR exposure. Data outside the 95% confidence interval of the mean was excluded. Repeat measure ANOVA testing (SigmaStat Statistical Analysis Program, SPSS Inc., Chicago, Ill.) was used to test for an effect due to post IR cofactor treatment with and without P188. If differences existed, Bonferroni's t-test was used to determine statistical significance. Statistical significance was defined as P values <0.05.

ii. Results

18 hr Viability of IR Exposed Cells Following Addition of Cofactors (Mg-ATP+NAC) with and without P188.

Experiments (Lee, Greenebaum et al., 2004) testing examining the viability of cells exposed to 40 Gy and subsequently treated with 1 mM P188 are shown in Table 1A. As demonstrated, the viability of cells treated solely with P188 is 20.6% at 18 hours compared to sham-exposed viability of 77.0% at 18 hours.

Our methodologies for determining the 18 hour and 48 hour survival of IR exposed cells was the exact same protocol employed by Lee and Greenebaum. Table 1B demonstrates the viability of IR-exposed cells treated with 10 mM NAC+0.1 mM Mg-ATP with and without 1 mM P188 (20.6%±3.3). At 18 hours, the mean percent survival of cells treated with NAC+Mg-ATP was (48.2%±6.0), dramatically greater than IR exposed cells that did not receive the cofactor treatment. The improved viability versus untreated IR-exposed samples was even more pronounced (55.2%±2.8) when P188 was added to the cofactors. Additionally, the viability of cofactor treated cells with and without P188 was significantly greater than those treated with 1 mM P188 alone (p<0.01).

48 hr Viability of IR Exposed Cells Following Addition of Cofactors (Mg-ATP+NAC) with and without P188.

Using the same experimental methodologies as above, we examined the viability of cofactor-treated cells with and without addition of P188 at 48 hours following irradiation. Cells that received both cofactor and P188 demonstrated statistically significant improved survival (29.0%±2.3) versus irradiated cells receiving no treatment (8.6%±2.1). Irradiated cells treated with cofactor alone also showed an increased survival versus those receiving no treatment (19.9% vs. 2.9%). Additionally, the group treated with cofactor and P188 appear to have better survival than those treated with cofactors alone (p<0.05).

iii. Discussion

The short term death of cells exposed to high doses of radiation (>10 Gy) is believed to be mediated via production of reactive oxygen intermediates. These species result in the peroxidation of membrane lipids, increasing its permeability. Results from prior studies indicate that P188 helps prevent short term cellular death following irradiation by sealing the lipid membrane. This reduces drastic changes in ion concentrations, thereby preventing massive ATP loss and cell death. Our results strongly suggest that the efficacy of P188 treatments can be enhanced with the addition of a cofactor treatment of N-acetylcysteine (Antioxidant) and MgCl₂-ATP (cellular energy source). We reason that NAC, an antioxidant supplies a reducing medium which the cell may use to neutralize ROIs. The Mg Cl₂-ATP serves to help replenish the energy sources lost by the cell while attempting to maintain its ionic gradients. Addition of these three compounds to irradiated cells results in an 18 hour viability that is nearly commensurate with cells that received no radiation treatment.

At 48 hours, the mean survival of cells exposed to radiation drops precipitously from survival at 18 hours. This finding may suggest that factors other than increased membrane permeability may contribute to cell death after 18 hours. In particular, this timeline appears to be consistent with an apoptotic model of cell death. Alternatively, the long-term drop in cell survival may reflect depletion of the cofactors over time as the cell uses them to maintain itself following irradiation.

TABLE 1A

| | No Radiation | 18 hrs Post-Radiation (40Gy) | |
|---|---|---|---|
| | 18 hr Control | No Treatment | P188 |
| Mean Survival | 77% ± 2.2 | 3.70% ± 1.2 | 20.60% ± 3.3 |

Table 1A demonstrates the 18 hr mean percent survival (±SEM) of sham-IR exposed cells, as well as survival of IR-exposed cells receiving and not receiving P188 treatment. Survival of cells that received P188 following irradiation was significantly improved versus those that received no treatment. However, the survival of cells treated with P188 remained substantially lower than cells that received no radiation exposure. (Lee R C, Greenebaum B, et al., 2004.)

TABLE 1B

| | No Radiation | 18 hrs Post-Radiation (40Gy) | |
|---|---|---|---|
| | 18 hr Control | No Treatment | Treatment |
| 10 mM | 78% ± 2.3 | 6.90% ± 3.0 | 48.20% ± 6.0 |
| NAC + 0.1 mM | | | |
| Mg-ATP | | | |
| 1 mM | 77.30% ± 1.8 | 6.80% ± 1.7 | 55.20% ± 2.8 |
| P188 + 10 mM | | | |
| NAC + 0.1 mM | | | |
| Mg-ATP | | | |

Table 1B: The 18 hr viability of cells receiving cofactors Mg-ATP and NAC with and without P188 treatment following 40 Gy exposure are shown. Survival is significantly improved among cells receiving the cofactor treatment versus control-irradiated cells. Additionally, cells receiving the cofactor treatment had significantly higher survival than cells in receiving strictly P188 (Table 1A). Cells that received P188 in addition to the cofactors had further improvement in survival.

TABLE 2

| | No Radiation | 48 hrs Post-Radiation (40Gy) | |
|---|---|---|---|
| | 48 hr Control | No Treatment | Treatment |
| 1 mM P188 | 85.30% ± 1.2 | 2.90% ± 1.3 | 6.90% ± 2.1 |
| 10 mM | 83.30% ± 1.6 | 2.90% ± 2.5 | 19.90% ± 2.9 |
| NAC + 0.1 mM | | | |
| Mg-ATP | | | |
| 1 mM | 82.70% ± 1.6 | 8.60% ± 2.1 | 29.00% ± 2.3 |
| P188 + 10 mM | | | |
| NAC + 0.1 mM | | | |
| Mg-ATP | | | |

Table 2: Shown are the survival of cells 48 hrs post 40 Gy irradiation treated with P188, cofactors, or a combination of the two. Cofactor treatment of cells immediately following irradiation significantly increased survival of cells at 48 hrs. Cells that received treatment with a combination of P188 and the cofactor treatment had significantly better survival (P>0.05) than those receiving cofactor treatment alone. The viability of cells at 48 hrs fell dramatically from the survival observed at 18 hrs.

F. Human Treatment Protocol

The following examples disclose contemplated treatment methods for human subjects from cellular membrane injury resulting in cellular membrane peroxidation. Representative events leading to cellular membrane injury can include, for example, colic, acute myocardial infarction, ischemia/reperfusion injury, cerebral palsy, muscular dystrophy, stroke, spinal cord injury, head injury, organ transplantation, inflammatory bowel conditions, cancer, severe infectious disease, necrotizing endocolitis, bacterial translocation, exposure to extreme thermal conditions such as frostbite or burns, conditions characterized by exposure to ionizing radiation and conditions characterized by exposure to chemical oxidants. Administration may be repeated daily as appropriate depending upon the severity of the cellular membrane injury and the response of individual to membrane sealing surfactant/cofactor treatment.

In certain representative embodiments, it is proposed that therapeutic compositions of the invention comprise a pharmaceutically appropriate carrier such as, for example, sterile water or buffered saline, a membrane sealing surfactant and a cofactor treatment including a cellular energy source and an antioxidant. A representative membrane sealing surfactant can comprise poloxamer P188 (available from BASF Co. of Parsippany, N.J. or as a formulation of poloxamer P188 called Rheo-thRX available from CytRx Corporation of Atlanta, Ga.) in a therapeutically effective amount from about 0.1 to about 5.0 mg/ml blood volume for repairing the cellular membrane. A representative antioxidant can comprise N-acetyl cysteine (NAC) in a therapeutically effective amount from about 25 mg to about 1000 mg for the purposes of reducing and/or eliminating the generation of Reactive Oxygen intermediates and enhancing metabolism of free radicals. A representative cellular energy source can comprise ATP supplied as MgCl₂-ATP in a therapeutically effective amount from about 0.1% to 15% w/v for re-establishing the cellular energy charge and restoring the cellular ion balance. A person of ordinary skill in the art will realize that additional ranges of membrane sealing surfactant, antioxidant and cellular energy source amounts are contemplated and are within the present disclosure. The presently contemplated therapeutic compositions can be injected either into a suitable vein or intramuscularly.

In certain representative embodiments, proposed therapeutic compositions of the invention comprise a topical application comprising a pharmacologically appropriate substrate having a membrane sealing surfactant at concentration from about 1.0% to about 10.0% w/v and a cofactor comprising a cellular energy source such as, for example, ATP in an amount form about 0.1% to about 15% w/v and an antioxidant such as, for example, N-acetyl cysteine (NAC) in an amount from about 25 mg to about 1000 mg. The topical application can be applied to the damaged area, wrapped as appropriate with sterile dressings, and reapplied as necessary.

In some representative embodiments, therapeutic treatments can comprise dual administration of a therapeutic composition such as, for example, combined administration of two or more suitable intravenous, intramuscular or topical compositions.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be achieved. All such similar substitutes and modifications apparent to those skilled in the art are deemed to be within the spirit, scope and concept of the invention as defined by the appended claims.

### REFERENCES

The references listed below are incorporated herein by reference to the extent that they supplement, explain, provide a background, or teach methodology, techniques, and/or compositions employed herein.
1995. Eur. J. Pharm. Sci. 3:39-48
Aeba et al. 1992. J. Thorac. Cardiovasc. Surg. 104:1333-9
Ahmed et al. 2000. J. Neurochem. 74: 1951-1960
Al-Khadra et al. 2000. Circ. Res. 87:797-804
Barnett et al. 1996. Stroke 27: 588-592
Becker 2004. Card. Res. 61:461-470
Bramlett et al. 2004. J. Cerebral Blood Flow & Metabolism. 24:133-150
Check and Hunter, “The Scientific Basis for the Biologic Activities of RheothRx™ Copolymer: A Rheologic, Antithrombotic and Cytoprotective Preparation,” CytRx Corp., 1988
Coghlan et al. 1994. J. Thorac. Cardiovasc. Surg. 107:248
Cosenza et al. 1994 Hepatology 19:418-425
Euler, 1995. Am. J. Physiol. 268:H295
Flaherty et al., 1994. Circulation 89:1982
Flahery, 1991. Am J. Med. 91:79S
Gonzalez et al. 1994. Transplantation 58:403-408
Granger et al. 1986. Acta Physiol. Scand. 47:S548
Hacke et al. 1995. JAMA 42:976-982
Hall et al. 1988. Stroke 19:997-1002
Hannig et al. 1999. IEEE Tran. Plasma Sci. 28:97-101
Hannig et al. 1999. Int. J. Radiat. Bio. 75:379-385
Hannig et al. 2000. Radiat. Res. 154:171-177
Hart and Harrison, 1996. Stroke 27:585-587).
Hormes et al. 1991. J. Stroke Cereborvasc. Dis. 1:27-35
Hossmann, 1994. Ann. Neurol. 36:557-65
Husain et al. 2002. BBA Biochem. et. Biphysic. Acta 1587: 75-82
Jone et al. 1981. J. Neurosurgery 54:773-782
Kaplan et al. 1991. Stroke 22:1032-1039
Kassell et al. 1990. J. Neurosurg. 73:18-36
Katz et al. 1995. Transplantation 59:694-8
Koptinik and Kaufman, 1992. Neurosurg. Clin. North Am. 3: 703-8
Kurihara et al. 1989. J. Allery Clin. Immunol. 83:83-90
Land et al. 1994. Transplantation 57:211
Lee. 2002. Ann. N.Y. Acad. Sci. 961:271-275
Lenzi et al. 1994 Stroke 25:1552-1558.
Levitt et al. 1994. J. Cardiovasc. Pharmacol. 23:136-140
Marks et al. 2001. FASEB J. Online Publication Feb. 20, 2001
McNeil et al. 1997. J. Cell Bio. 137:1-4
Mueller et al. 1994. Transplantation 58: 1309-1316
Nalini et al. 1993. Mol Cell. Biochem. 124:59-66
Palmer et al. 1997. Pediatrics 100:S571, 99
Park et al. 1994. Arch. Surg. 129:857-600
Parks and Granger, 1986. Am. J. Physiol. 250 (6 Pt 1):749-53
Parks et al. 1982. Gastroenterology 82:9-15
Parmley, 1991. Can. J. Cardiol. 8:280
Pollack et al. 1993. Transplantation 55:57
Rashid and Goran, 1991. Ann. Thorac. Surg. 52:127
Shackleton et al. 1994. J. Surg. Res. 57: 433-7
Stone et al. 1992. Am. J. Vet. Res. 53: 2153-6
Terry et al. Ann. 1999. N.Y. Acad. Sci. 888:274-284′
Van Ye et al. 1993. J. Surg. Res. 55:553-8
Vanhaecke, 1991. J. Am. Coll. Cardio. 18:224
Yang and Betz, 1994. Stroke 25:1658-1665
Yasuda et al. 2005. nature. Doi:10.1038/nature03844

What is claimed is:

1. A method for increasing cell viability following exposure of mammalian cells to an event resulting in cellular membrane peroxidation comprising:
delivering to cells a therapeutic composition comprising a pharmaceutically acceptable carrier, a membrane sealing surfactant and a co-factor treatment, the co-factor treatment consisting of an antioxidant and a high energy phosphate compound, wherein application of the therapeutic composition to the exposed mammalian cells increases cell viability at a time 18 hours subsequent to the event by statistically significant amount upon application of the pharmaceutical composition to the exposed mammalian cells when compared to individual application of the membrane sealing surfactant or the co-factor treatment to the exposed mammalian cells.

2. The method of claim 1, wherein application of the therapeutic composition to the exposed mammalian cells increases cell viability at a time 18 hours subsequent to the systemic event by at least 10% upon application of the pharmaceutical composition to the peroxidized cells when compared to individual application of the membrane sealing surfactant or the co-factor treatment to the peroxidized cells.

3. The method of claim 1, wherein application of the therapeutic composition to the peroxidized cells comprises in vivo application of the therapeutic composition.

4. The method of claim 3, wherein in vivo application of the therapeutic composition comprises application of the membrane sealing surfactant at a level from about 0.01 to about 5.0 mg/ml blood volume.

5. The method of claim 4, wherein in vivo application of the therapeutic composition comprises application of the membrane sealing surfactant at a level from about 0.1 to about 5.0 mg/ml blood volume.

6. The method of claim 1, wherein the event is selected from the group comprising: colic, acute myocardial infarction, ischemia/reperfusion injury, cerebral palsy, muscular dystrophy, stroke, spinal cord injury, head injury, organ transplantation, necrotizing endocolitis, bacterial translocation, exposure to ionizing radiation and exposure to chemical oxidants.

7. The method of claim 1, wherein the membrane sealing surfactant is selected from the group comprising: a poloxamer, a meroxapols, a poloxamine, a PLURADOT™ polyol and combinations thereof.

8. The method of claim 7, wherein the membrane sealing surfactant comprises poloxamer P188.

9. The method of claim 1, wherein the antioxidant is selected from the group comprising: ascorbic acid, tocopherol, Vitamin A, mannitol, a bioflavonid, a flavonoid, a flavone, a flavonol, proanthocyanidin, selenium, gluthathione, N-acetyl-cysteine, superoxide dismutase, lipoic acid, coenzyme Q-10, beta-carotene, lycopene, lutein, polyphenol and combinations thereof.

10. The method of claim 1, wherein the high energy phosphate is selected from the group comprising: adenosine triphosphate, adenosine diphosphate, phosphocreatine and combinations thereof.

11. The method of claim 10, wherein the high energy phosphate comprises MgCl₂-ATP.

12. A therapeutic composition for treating mammalian cells exposed to a peroxidation event comprising:
a pharmaceutically acceptable carrier;
a membrane sealing surfactant; and
a co-factor treatment, the co-factor treatment having an antioxidant and a cellular energy source,
wherein application of a therapeutically effective amount of the therapeutic composition to the exposed mammalian cells increases cell viability at a time 18 hours subsequent to a peroxidation event by at least 10% when compared to individual application of the membrane sealing surfactant or the co-factor treatment to the exposed mammalian cells.

13. The therapeutic composition of claim 12, wherein application of the therapeutic composition to the exposed mammalian cells increases cell viability at a time 48 hours subsequent to the peroxidation event by at least 40% when compared to individual application of the membrane sealing surfactant or the co-factor treatment to the exposed mammalian cells.

14. The therapeutic composition of claim 12, wherein the membrane sealing surfactant is selected from the group comprising: a poloxamer, a meroxapols, a poloxamine, a PLURADOT™ polyol and combinations thereof.

15. The therapeutic composition of claim 14, wherein the membrane sealing surfactant comprises poloxamer P188 in an amount for a particular subject to result in a concentration from about 0.01 to about 5.0 mg/ml blood volume.

16. The therapeutic composition of claim 12, wherein the antioxidant is selected from the group comprising: ascorbic acid, tocopherol, Vitamin A, mannitol, a bioflavonid, a flavonoid, a flavone, a flavonol, proanthocyanidin, selenium, gluthathione, N-acetyl-cysteine, superoxide dismutase, lipoic acid, coenzyme Q-10, beta-carotene, lycopene, lutein, polyphenol and combinations thereof.

17. The therapeutic composition of claim 16, wherein the antioxidant is N-acetyl-cysteine in an amount from about 25 mg to about 1000 mg.

18. The therapeutic composition of claim 12, wherein the cellular energy source is selected from the group comprising: adenosine triphosphate, adenosine diphosphate, phosphocreatine and combinations thereof.

19. The therapeutic composition of claim 18, wherein the cellular energy source comprises MgCl₂-ATP in an amount from about 0.1% to 1.0 w/v.
